(19) United States
(12) Patent Application Publication
(10) Pub. No.: US 2015/0351735 A1
(43) Pub. Date: Dec. 10, 2015

(54) Temporary Atrium Support Device

(71) Applicant: MEDTENTA INTERNATIONAL LTD OY, Helsinki (FI)

(72) Inventors: Olli Keränen, Bjärred (SE); Jani Virtanen, Sipoo (FI); Mark Pugh, Coolaney, Co. Sligo (IE); Ger O'Carroll, Co. Sligo (IE); Adrian Moran, Co. Sligo (IE)

(73) Assignee: MEDTENTIA INTERNATIONAL LTD OY, Helsinki (FI)

(21) Appl. No.: 14/762,437

(22) PCT Filed: Jan. 27, 2014
(86) PCT No.: PCT/EP2014/051542

§ 371 Date: Jul. 21, 2015

Related U.S. Application Data
(60) Provisional application No. 61/756,663, filed on Jan. 25, 2013.

(30) Foreign Application Priority Data
Jan. 25, 2013 (EP) 13152771.5

(51) Int. Cl. A61B 17/02; A61F 2/24

(52) U.S. Cl. CPC A61B 17/0218; A61F 2/2487; A61B 2017/0225; A61B 2017/00867

(57) ABSTRACT

A temporary expandable and contractible atrium supporting device (100), comprising an expandable and contractible intra atrial support member (101) being resiliently flexible to allow for atrium (2) contraction and expansion, when positioned intra atrial substantially maintaining atrial displacement volume of the beating heart.

### RELATED APPLICATIONS

This application is related to application EP13152770.7, U.S. 61/756,649 “A valve for short time replacement, for taking over the function of and/or for temporary or partial support of a native valve in a heart” filed 25 Jan. 2013, application EP13152769.9, U.S. 61/756,657 “A medical system, and a device for collecting chordae and/or leaflets” filed 25 Jan. 2013, application EP13152774.9, U.S. 61/756,633 “A medical device and method for facilitating selection of an annuloplasty implant” filed 25 Jan. 2013 and application EP13152768.1, U.S. 61/756,670 “A system for cardiac valve repair” filed 25 Jan. 2013.

### BACKGROUND OF THE INVENTION

1. Field of the Invention

This invention pertains in general to the field of medical devices for improvement of an atrium. More particularly the invention relates to a temporary atrium support device and a method of delivery therefor.

2. Description of the Prior Art

During heart surgery at an atrium or related to the atrium, where the function of the atrium need to be or is reduced, there is an increased risk of the atrium collapsing. The collapse of the atrium may be induced by a variety of factors such as a pressure drop in the atrium, and/or a structural deficiency of the atrium. The pressure drop in the atrium may be due to a procedure of implanting a valve or other procedures resulting in a reduced pressure in the atrium.

A collapse of the atrium is highly undesired because it strongly influences the functioning of the heart and thus affects a patient in a non desired way. During surgery or intervention this means additional effort and personal is needed to maintain and monitor the atrium keeping the atrium functioning normally.

Hence, there is a strong need for preventing the collapse of the atrium to secure proper functioning of the heart and thus providing space for mitral intervention and the well being of a patient.

Prior art devices are insufficient in providing such functions.

### SUMMARY OF THE INVENTION

Accordingly, examples of the present invention preferably seek to mitigate, alleviate or eliminate one or more deficiencies, disadvantages or issues in the art, such as the above-identified, singly or in any combination by providing an temporary atrium support device and a method of delivery therefor, according to the appended patent claims.

According to aspects of the disclosure, an atrium support device and a method of delivery therefor are disclosed.

According to a first aspect of the disclosure, a temporary atrium supporting device is provided. The atrium support device comprises an expandable and contractible intra-atrial support member being resiliently flexible to allow for atrium contraction and expansion, when positioned intra-atrial substantially maintaining atrial displacement volume of the beating heart. By using a temporary atrium support device a collapse of the atrium is prevented. The collapse of the atrium could be a consequence of an atrium pressure drop introduced by e.g. performing a repair of a mitral valve and/or other procedures performed in relation with the functioning of the atrium. The atrium support device thus secures the function of the atrium and consequently a heart during a medical procedure performed on the heart.

According to a second aspect of the disclosure, a method of temporary preventing atrial collapse in a beating heart is provided. The method comprises intra-atrial positioning an expandable and contractible atrium support device, the atrium support device being resiliently flexible when expanded, and expanding the atrium support device in the atrium thus keeping the atrium non-collapsed, and allowing contraction and expansion of the non-collapsed atrium by the atrium support device.

Further examples are defined in the dependent claims, wherein features for the second and subsequent aspects of the disclosure are as for the first aspect mutatis mutandis.

Some examples of the disclosure provide for an atrium support member having a volume that never is smaller than a predefined volume.

Some examples of the disclosure provide for an atrium support member securing that there is a minimum of desired blood present in the atrium.

Some examples of the disclosure provide for a minimum blood flow through the heart is ensured.

Some examples of the disclosure provide for a beating heart and/or heart support equipment to maintain a minimum circulation of blood in a patient.

Some examples of the disclosure provide for a flexibility of an atrium support member to result in a maximum volume of the atrium support member.

Some examples of the disclosure provide an atrium to be controlled to result in a maximum atrium volume.

Some examples of the disclosure provide for aiding in an atriums reshaping to resume a maximum volume during relaxation of the atrium.

Some examples of the disclosure provide for reducing a damage of an atrium by over-expansion.

Some examples of the disclosure provide for an expansion of an atrium support member to be performed in a variety of ways.

Some examples of the disclosure provide for an atrium support device to be comprised of a wide selection of materials capable of being temporary introduced into the atrium.

Some examples of the disclosure provide for an atrium support device which does not induce any damages to the catheter and/or the atrium.

Some examples of the disclosure provide for expansion of an atrium support member in a controlled and faster way.

Some examples of the disclosure provide for better customization of an atrium support device to better suit a shape of an atrium.

Some examples of the disclosure provide for an atrium support device which easily is deployed trough a catheter.

Some examples of the disclosure provide for aligning and securing an atrium support device.

Some examples of the disclosure provide for an atrium support device remaining substantially at a same location during an expansion and a contraction of a heart.

Some examples of the disclosure provide for no unnecessary damages occurring due to any rotation and/or twisting of an atrium support member.

Some examples of the disclosure provide for blood flow to be secured between the pulmonary vein and the mitral valve.

Some examples of the disclosure provide for a simple but yet effective alignment of an atrium support device.

Some examples of the disclosure provide for partial support of an atrium.

Some examples of the disclosure provide for maximum support and prevention of collapse of an atrium.

Some examples of the disclosure provide for customization of the overall shape and size of an atrium support device to better adapt to a shape of an atrium.

Some examples of the disclosure provide for better compliance of an atrium support device with an expansion and contraction of the atrium during beating of a heart.

Some examples of the disclosure provide for deploying an annuloplasty device to a heart valve.

Some examples of the disclosure provide for faster attachment of an annuloplasty device.

Some examples of the disclosure provide for a simple yet effective solution for guiding an annuloplasty device into place.

Some examples of the disclosure provide for protection of tissue and/or other parts of an atrium when positioning an annuloplasty device.

Some examples of the disclosure provide for a minimum of strain to be exerted to an annuloplasty device when deployed at a desired heart location.

Some examples of the disclosure provide for a more accurate and simple guiding of an annuloplasty device to the desired heart site.

Some examples of the disclosure provide for a positioning of an atrium support device through an existing opening into an atrium.

Some examples of the disclosure provide for a positioning of an atrium support device to result in a minimum of damage of an atrium.

Some examples of the disclosure provide for a possibility to introduce other tools and/or devices through existing natural openings into an atrium.

Some examples of the disclosure provide for reducing any complications of a leak if an atrium support device into an atrium.

Some examples of the disclosure provide for accessible means during a heart surgery to inflate an atrium support member.

The meaning of atrial displacement in this application should be understood to mean the throughput of blood through the atrium and to provide space for mitral valve intervention.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which examples of the disclosure are capable of will be apparent and elucidated from the following description of examples of the present disclosure, reference being made to the accompanying drawings.

FIG. 1 is a cross sectional view of a heart with an expanded atrium support member;

FIG. 2 is a cross sectional view of a heart with an atrium support member positioned with a catheter through a mitral valve;

FIG. 3 is a cross sectional view of a heart with an expanded atrium support member deployed with a catheter through a mitral valve;

FIG. 4 is a cross sectional view of an atrium with an expanded atrium support member positioned through an aorta.

FIG. 5 is a perspective view of an expanded atrium support member comprising means for aligning;

FIG. 6 is a cross sectional view of an expanded atrium support member comprising a channel through the atrium support member from a pulmonary vein to a mitral valve;

FIG. 7 is a perspective view of an expanded atrium support member comprising guiding means at an outer surface.

### DESCRIPTION OF THE PREFERRED EXAMPLES

Specific examples of the disclosure will now be described with reference to the accompanying drawings. This invention may, however, be embodied in many different forms and should not be construed as limited to the examples set forth herein; rather, these examples are provided so that this disclosure will be thorough and complete, and will fully convey the scope of the invention as defined by the appended claims to those skilled in the art. The terminology used in the detailed description of the examples illustrated in the accompanying drawings is not intended to be limiting of the invention. In the drawings, like numbers refer to like elements.

The following description focuses on an example of the present disclosure applicable to a support device and in particular to a support device for an atrium. However, it will be appreciated that the disclosure is not limited to this application but may be applied to many other areas in a body where support is needed.

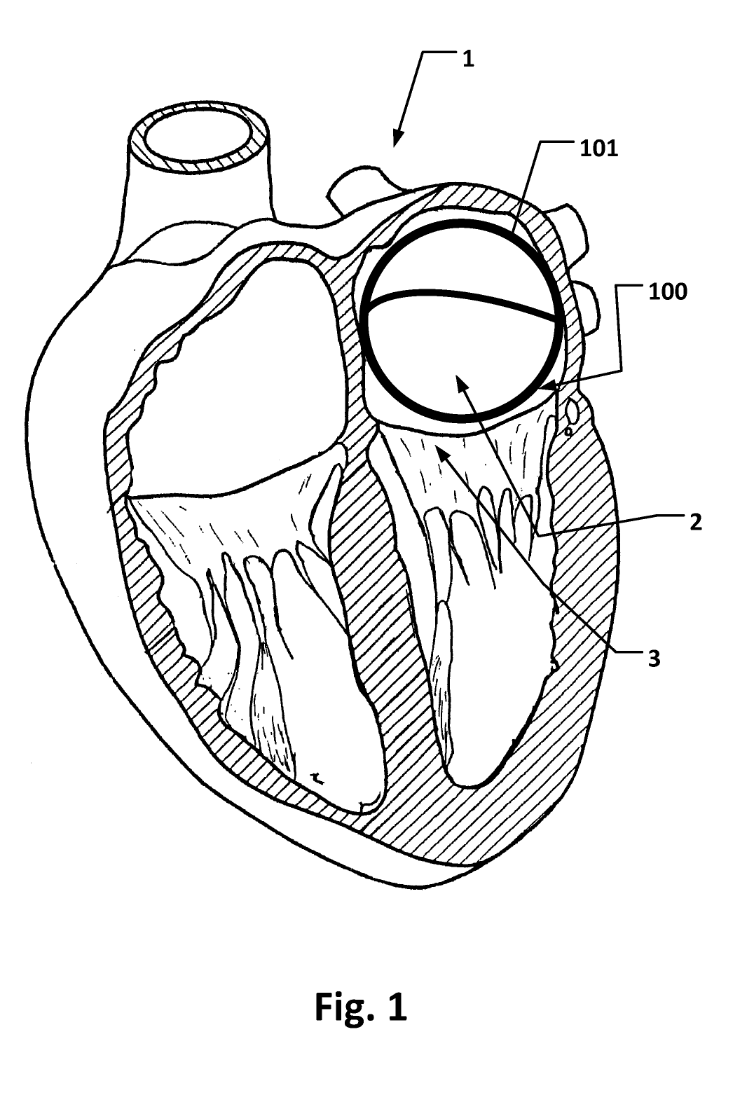

In an example of the disclosure according to FIG. 1, a temporary expandable and contractible atrium supporting device 100 positioned inside an atrium 2 in a heart 1 is shown. The atrium supporting device 100 comprises an expandable and contractible intra atrial support member 101. The support member 101 is resiliently flexible to allow for atrium 2 contraction and expansion, when positioned intra atrial. The support member 101 substantially maintains atrial displacement volume of the beating heart. By using a temporary atrium support device 100 a collapse of the atrium 2 is prevented. The collapse of the atrium 2 could be a consequence of a pressure drop in the atrium 2 introduced by e.g. when performing a repair of a mitral valve 3 and/or other procedures performed in relation with the functioning of the atrium 2. The atrium support device 100 thus secures the function of the atrium 2 and consequently the heart during a procedure which is related to the atrium 2 and/or the atrium 2 itself. Further, by being resiliently flexible to allow for natural atrium contraction and expansion while maintaining an atrial displacement volume, disturbance of the heart function is minimized. Other devices, such as those placed in the atrium for occlusion of defects, or re-shaping devices, or other devices for permanent anchoring provide too high force to the anatomy, or permanent damage to the tissue, due to their different purpose, and consequently disturb the natural heart function significantly. Also, devices for lining or coating portions of the heart to prevent blood cloths does not provide any support for the atrium to maintain a displacement volume of a collapsing atrium. On contrary, the atrium support member of the present disclosure improve the compatibility and synchronous function with the heart due to being resiliently flexible its expanded state to follow the natural beating movement.

In order to maintain a normal or an adequate function of the atrium 2 the resilient flexibility of the atrium support member 101 results in an atrium volume that is preferably more than 55 ml, more preferably more than 50 ml, even more preferably more than 20 ml and most preferably more than 15 ml. By having the atrium support member 101 resiliently flexible such that the atrium support member 101 has a volume that may be changed and that never is smaller than a predefined volume, the atrium support member 101 secures that there is a minimum of desired blood present in the atrium 2 and a minimum blood flow is thus ensured. This allows for a beating heart and/or heart support equipment to maintain a minimum circulation of blood in a patient. Further, the resilient flexibility of the atrium support member 101 results in an atrium volume that is preferably at the most 100 ml, more preferably at the most 90 ml, even more preferably at the most 80 ml and most preferably at the most 60 ml. By allowing the resiliently flexibility of the atrium support member 101 to obtain more than a maximum volume of the atrium 2 the atrium 2 is controlled to result in a maximum atrium volume. Additionally, by defining the flexibility of the atrium support member 101 to the maximum volume the atrium 2 is aided in its reshaping to resume the maximum volume during relaxation of the atrium 2. Further, by constraining the flexibility of the atrium support member 101 to the maximum volume, damage of the atrium 2 by over-expansion is reduced.

The atrium support member may thus have a predefined maximum expanded cross-section. The atrium support member may thus also have a predefined minimum contracted cross-section when placed in the atrium, such that the compressive force exerted by the atrium on the atrium support member at the minimum contracted cross-section is compensated and counter acted by a reaction force of the support member on the atrium, and where the reaction force is equal to that of said compressive force. The reaction force at the minimum contracted cross-section can thus be set to a pre-defined value. This can e.g. be done during heat setting procedures of the material to define its properties.

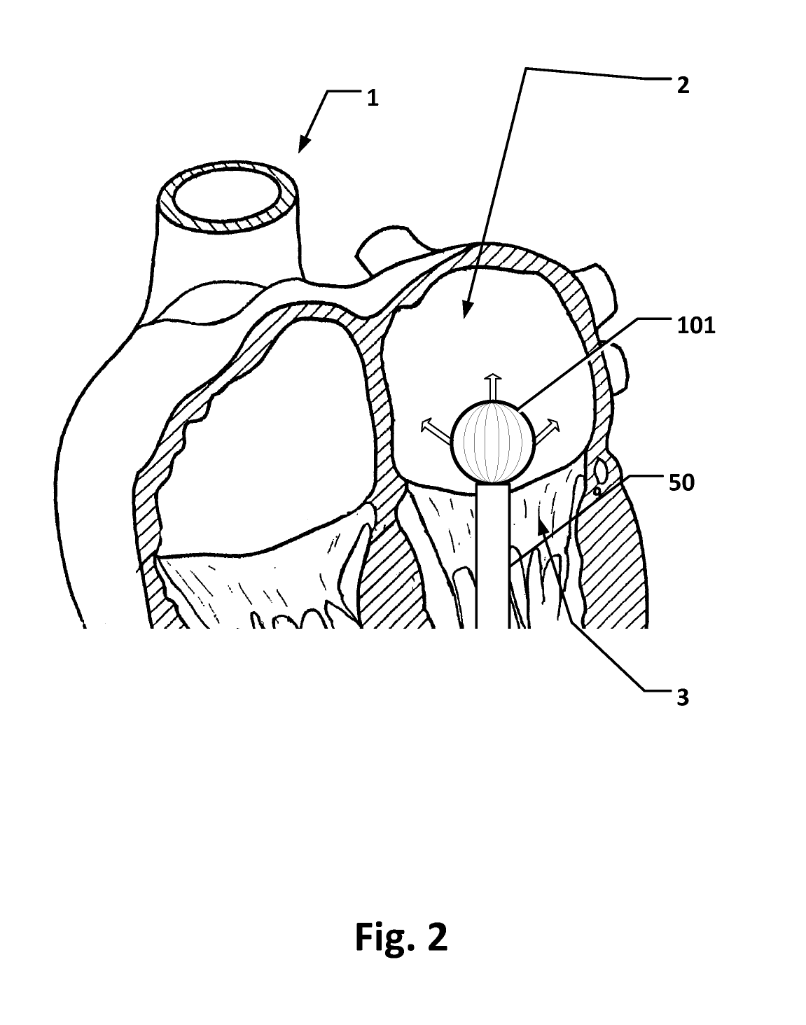

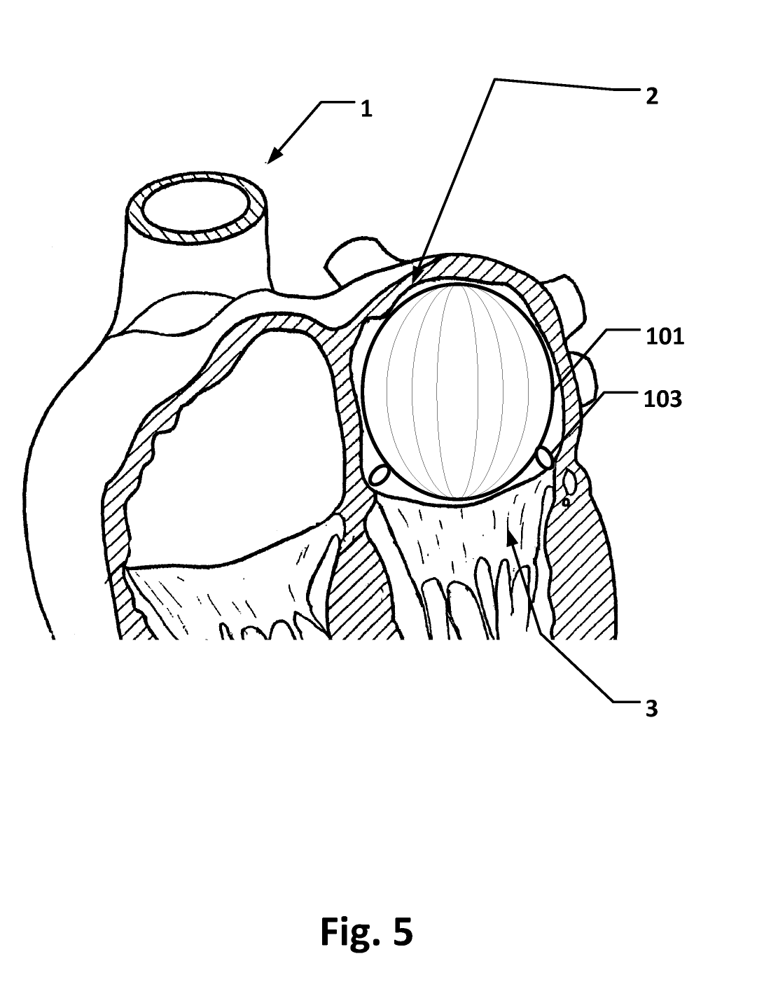

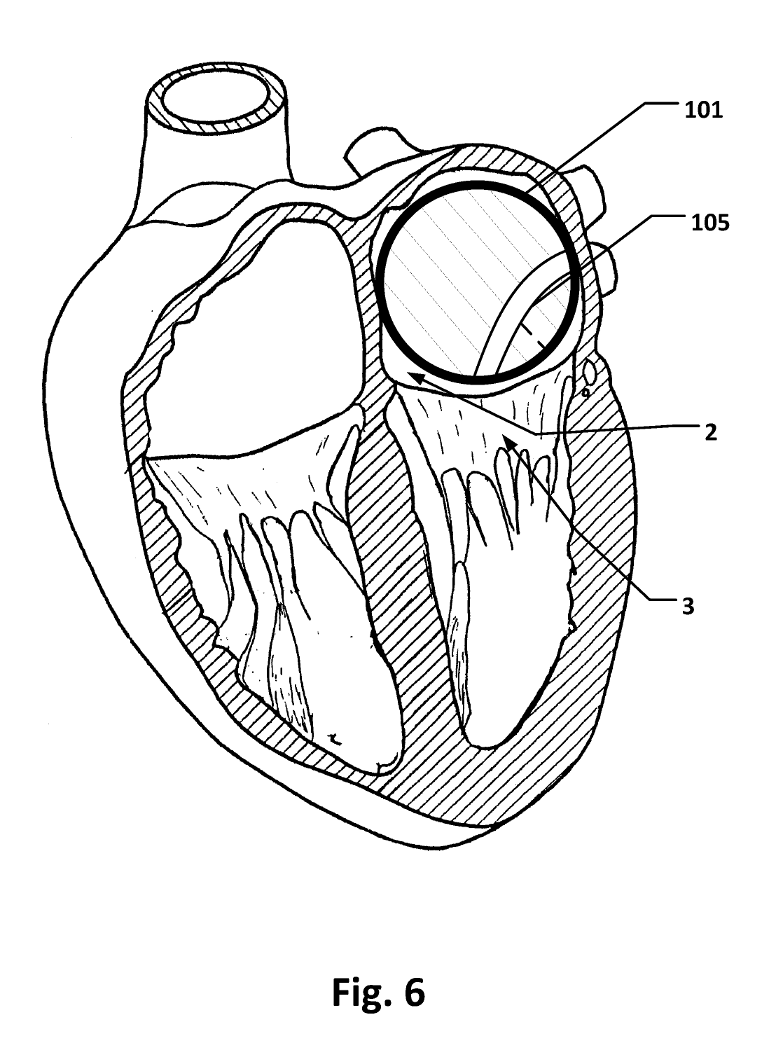

The atrium support member 101 is of a shape and material that is capable of being inserted and guided through a catheter 50 to the atrium 2 as illustrated in FIGS. 2-4. The atrium support member 101 may comprise an expandable cage, as illustrated in FIGS. 1-3, 5 and 7, and/or alternatively a wire, and/or an inflatable member comprising at least one channel 105, as illustrated in FIG. 6, and/or a covering comprising holes which allows for substantially maintaining the atrial displacement volume. In one example the expandable cage comprises at least two intersecting cage rings, but more preferably more than two intersecting cage rings in order to achieve more area of contact between the cage rings outer surface towards the atrium tissue and the atrium tissue. The atrium support member may comprise an inlet and outlet for blood flow. This assures that the natural flow of blood is not disturbed while maintaining the displacement volume.
In another example the at least one channel in the inflatable member comprises a valve. The valve may be of the cardiac valve type, i.e. being closed when the heart chamber ejects blood to the body or lungs respectively and open during a refill phase. The dimension of the at least one channel and any possible valve is chosen based on a wanted atrial displacement. The same applies for selection of the size of the holes in the covering.

The expansion of the atrium support member 101 may be performed in a variety of ways and the atrium support device 100 may be comprised of a wide selection of materials capable of being temporary introduced into the atrium 2 and being expandable from the catheter 50 without diverging from the scope of the invention. Following, some examples will be giving but they should not be construed as limiting.

The atrium support member 101 is preferably made of a material that is biocompatible and designed in such way that the atrium support member 101 does not induce any damages to the catheter 50 and/or the atrium 2.

For example the atrium support device 100 comprises a memory shape material, wherein the memory shape material has a first shape when deployed and a second expanded shape activated by a shape memory temperature. By using the atrium support device 100 comprising the shape memory material it is possible to activate the atrium support device 100 to expand to the second expanded shape in a controlled and faster way than with other expansion techniques. Further, the memory shape material allows for better customising the expanded shape of the atrium support device 100 to better suit the shape of the atrium 2. Suitable materials for the shape memory material are e.g. copper-aluminium-nickel alloys, nickel-titanium alloys and/or other known shape memory materials.

In yet another example the atrium support member 101 comprises a heat set shape, and wherein the atrium support member 101 elastically returns to the heat set shape. By use of the heat set shape it is possible to get an atrium support member 101 which has inherent elastic properties to a desired shape and can easily be deployed trough the catheter 50. Suitable materials for the heat set shape are e.g. nickel-titanium alloys and/or other known heat set shape materials.

In another example, the atrium support device 100 comprises and/or acts as, a leaflet limiter which limits abnormal movement, such as prolapse, of the leaflets into the atrium. Such abnormal movement may arise if a chordae, or several chordae, that usually limits the movement of the leaflet is completely destroyed and the leaflet may thus freely move in the left atrium and/or left chamber. The leaflet limiter is made of a material that can expand together with the atrium support device 100, and/or it may be made of the same material as the atrium support device 100. Alternatively, the leaflet limiter is expanded by a spring back when exited from the catheter 50. The leaflet limiter may be a crossbar that extends and is projected laterally from the atrium support device 100. The number of leaflet limiters and their placement is chosen based on the circumstance that the atrium support device 100 is used in and may thus be of a number of different shapes and have various placements. One example would be to have a simple projection outwards towards the leaflets from the atrium support device 100 that limits the movement or other suitable shapes that limits but not damage the leaflet(s) when hindering its movement into the atrium. Preferably, the atrium support device 100 has two leaflet limiters, one on each side of the atrium support device 100 for each leaflet. But, there could also be only one leaflet limiter if it is known that one leaflet is already damaged and moving freely when starting a procedure of deploying the atrium support device 100. As mentioned, the atrium support device itself may acts as a leaflet limiter when expanded in the atrium, and no other component may be required. This provides for a device that limits leaflet movement that is easy to handle and position.

In an example the atrium support device 100 comprises means for aligning 103 the atrium support device 100 by use of at least one commissure, wherein the means for aligning 103 comprises a first end and a second end, and wherein the first end of the means for aligning is connected to the atrium support member 101 and the second end is directed outwards from the atrium support member 101 towards the at least one commissure. The means for aligning the atrium support member 101 assists in aligning and securing the atrium support member 101 from rotation when the atrium 2 is e.g. relaxed with a larger volume than the atrium support member 101 and/or the atrium support member 101 comprises the inflatable member with the at least one channel 105. In one example, if the atrium 2 is relaxed with the larger volume than the atrium support member 101 the means for aligning is for securing and aligning the atrium support member 101 to the atrium 2 so that the atrium support member 101 remains substantially at the same location during the expansion and contraction of the heart and no unnecessary damages occurs due to any rotation and/or twisting of the atrium support member 101. In another example, if the atrium support member 101 comprises the inflatable member with at least one channel 105 which extends from the pulmonary vein to the mitral valve 3, the means for anchoring ensures that the atrium support member 101 aligns the channel 105 to the pulmonary vein and the mitral valve 3 during the expansion and contraction of the atrium 2 so that blood flow is secured between the pulmonary vein and the mitral valve 3.

The means for aligning 103 the atrium support member 101 is in one example a pair of projections 103, projecting outwards from the atrium support member 101. By using a pair of projections 103 for aligning the atrium support member 101, a simple but yet effective alignment of the device is achieved. For example the atrium support member 101 is easily aligned by simply using the pair of projections 103 at and/or into suitable aligning sites in the atrium 2 such as the commissures and/or the mitral valve 3 and the pulmonary vein.

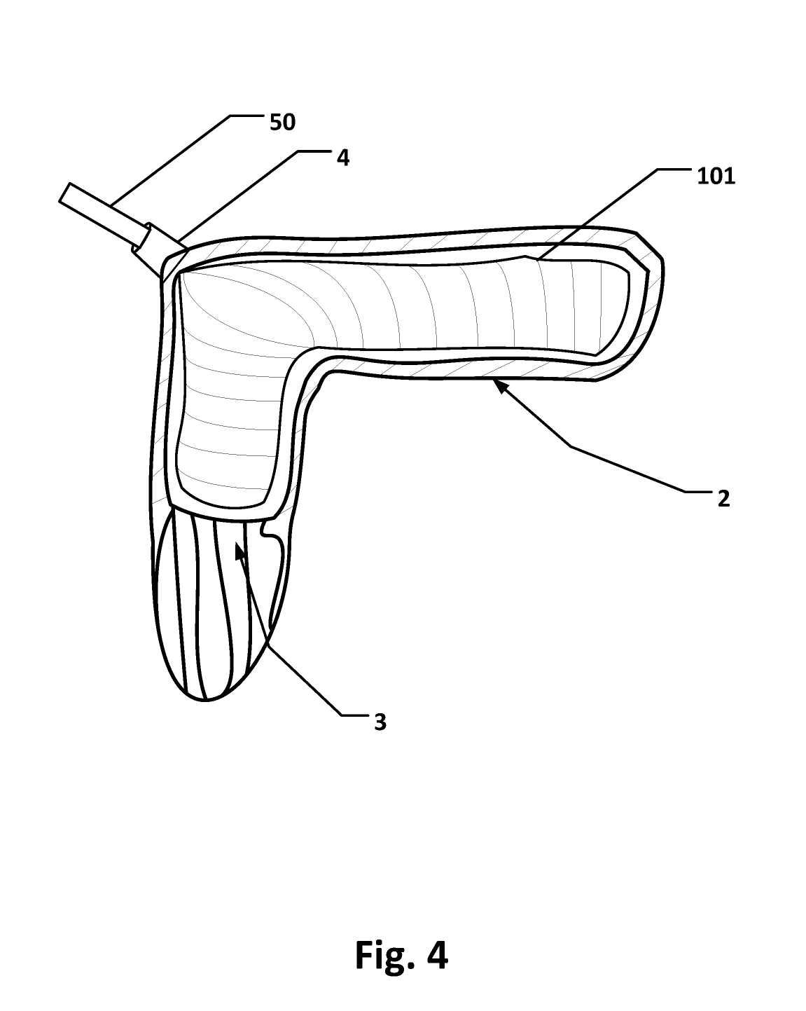

In some examples the atrium support member 101 is partly in contact with the atrium 2. By allowing the atrium support device 100 being partly in contact with the atrium 2 the support device is e.g. chosen to support a larger section of the atrium 2 or chosen to support a smaller section of the atrium 2. Alternatively, the atrium support member 101 is in contact with substantially the entire atrium 2 as illustrated in FIG. 4. By having the support device contacting the entire atrium 2, maximum support and prevention of collapse of the atrium 2 is ensured.

The atrium support member 101 may be designed in a variety of ways to provide partial contact or substantially full contact or get into apposition with the atrium 2. Such designs are e.g. the atrium support member 101 is bent when expanded, such as banana shaped as for instance in FIG. 4. The atrium support member 101 may be spherical when expanded. The atrium support member 101 may be bulb shaped when expanded. Alternatively, the atrium support device 100 comprises a plurality of atrium support member 101. By use of a plurality of atrium support members 101 it is possible to customise the overall shape and size of the atrium support device 100 to better adapt to the shape of the atrium 2 than a single atrium support member 101. Further, it would be possible to have atrium support members 101 with different flexibility at different locations in the atrium 2 which allows for better compliance of the atrium support device 100 with the expansion and contraction of the atrium 2 during beating of the heart.

In yet another example, the atrium support device 100 further comprises means for guiding 107 an annuloplasty device from an insertion site to a securing site at a heart valve. By having the atrium support device 100 further comprising guiding means 107 it is possible to aid the operator to in deploying the annuloplasty device to the heart valve. This allows for faster attachment of the annuloplasty device compared to when the operator need to introduce other equipment to secure the annuloplasty device.

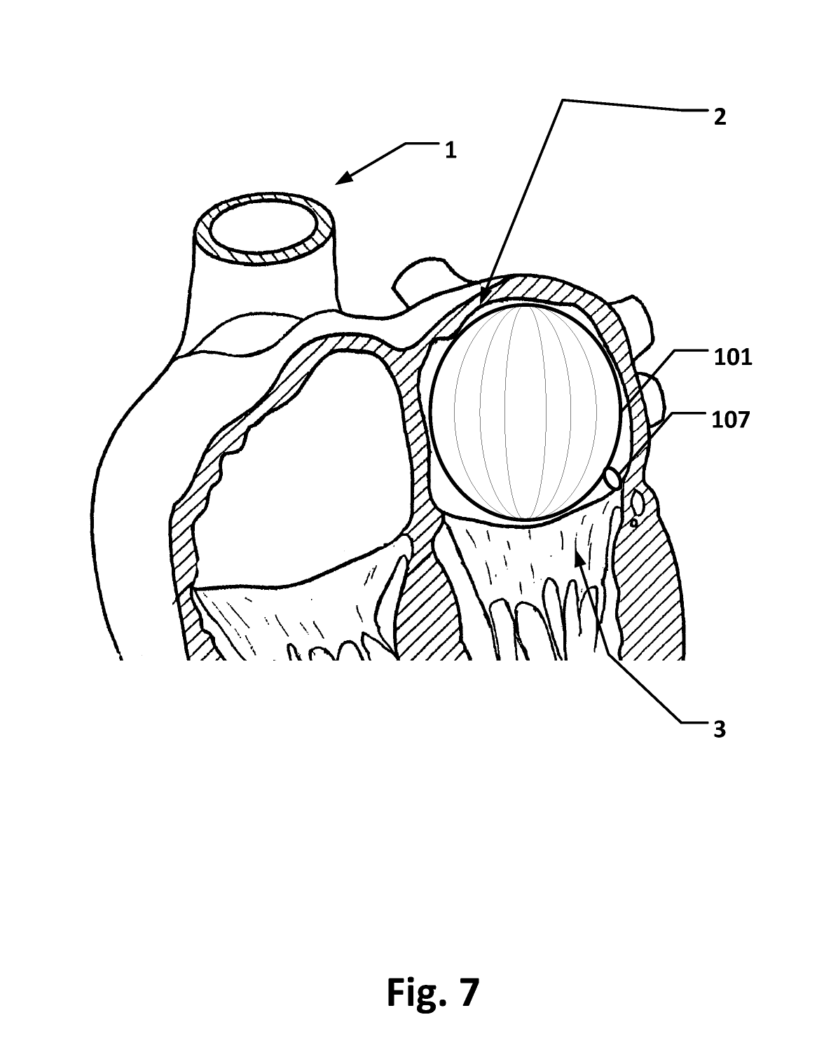

In one example the guiding means of the atrium support device 100 is at least one ring shaped member 107 arranged at an outer surface of the atrium support member 101 which is illustrated in FIG. 7. The use of at least one ring shaped member 107 provides a simple yet effective solution for guiding the annuloplasty device into place. The outer surface of the atrium support member 101 is the surface facing the tissue of the atrium 2. The arrangement of the at least one ring shaped member 107 on the outer surface of the atrium support member 101 is achieved by e.g. attaching the ring shaped member 107 at, on or through the outer surface.

In another example the means for guiding is a channel arranged along an outer surface of the atrium support member 101. The use of the channel as the guiding means is particularly beneficial when the atrium support member 101 is a sheet, covering or other shell shaped atrium support member 101. In such case the annuloplasty device is guided along the channel arranged along the outer surface of the atrium support device 100 to a desired heart site. The channel is preferably a coherent channel but may also be a sectioned channel.

In yet another example the means for guiding is a plurality of holes at the atrium support member 101. The use of holes in the atrium support member 101 the guiding of the annuloplasty device is performed through the atrium support member 101 which allows for the atrium support member 101 to protect the tissue and/or other parts of the atrium 2 when positioning the annuloplasty device. Additionally, the use of the plurality of holes provides guiding when the annuloplasty device has a smaller diameter than the atrium support member 101 and a minimum of strain is to be exerted to the annuloplasty device.

In yet an alternative example, the atrium support member 101 comprises e.g. the inflatable member or another substantially solid object, the means for guiding is at least one channel arranged through the atrium support member 101. The use of at least one channel through the atrium support member 101 provides the same solution for protecting the atrium 2 and exerting the minimum of strain as with the plurality of holes. In addition, the at least one channel through the atrium support member 101 ensures for a more accurate and simple guiding of the annuloplasty device to the desired heart site.

A further example of the disclosure is illustrated in FIGS. 2-4 where a method of temporary preventing atrial collapse in a beating heart, comprising intra atrial positioning an expandable and contractible atrium support device 100, the atrium support device 100 being resiliently flexible when expanded and expanding the atrium support device 100 intra atrial thus keeping the atrium 2 non-collapsed and allowing contraction and expansion of the non-collapsed atrium 2 by the atrium support device 100.

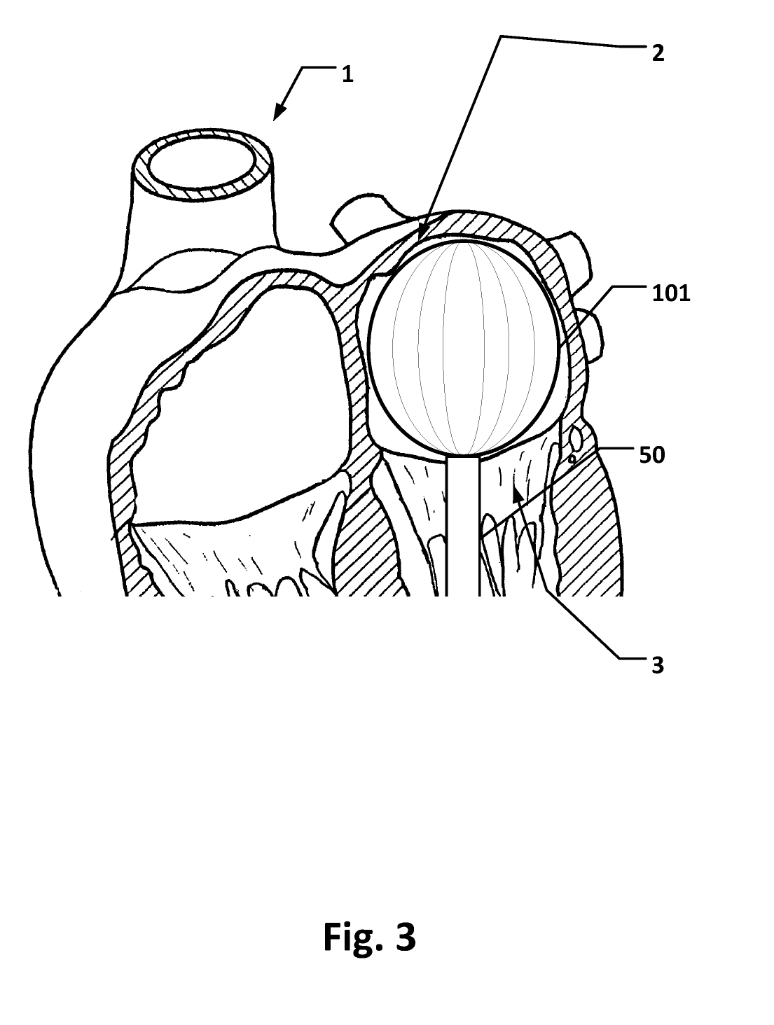

As illustrated in FIGS. 2-4 an example of the positioning of the atrium support device 100 is performed through an existing opening into the atrium 2. In one example the positioning is performed through the mitral valve 3 as illustrated in FIGS. 2-3, but other existing openings such as a pulmonary vein or an aorta 4, as illustrated in FIG. 4, can be used to introduce the atrium support device 100 into the atrium 2. By using the existing openings to the atrium 2 a minimum of damage of the atrium 2 is achieved. Well known procedures using these openings are transapically through the mitral valve 3 or trans-femorally through the aortic valve and the mitral valve 3 for entering the left atrium.

Alternatively in another example, the positioning of the atrium support device 100 is performed through an atrium wall. By having the atrium support device 100 positioned through the atrium wall it is possible to introduce other tools and/or devices through the existing openings. The existing openings may also be blocked and/or to narrow to introduce the atrium support device 100 resulting in that the operator is forced to go through the atrium wall and/or septum wall.

The expansion of the atrium support device 100 intra-atrial preventing the collapse of the atrium 2 is preferably performed by use of a force on the atrium support device 100. Such force may be a pulling force, a pushing force, an elastic force and/or an expansion force.

In another example the expansion of the atrium support device 100 is performed by use of applying a shape memory temperature to the atrium support device 100. By using the shape memory temperature to the atrium support device 100 the atrium support device is triggered to expand. The shape memory temperature is chosen to be triggered at e.g. a temperature of the blood in the atrium 2 or a temperature of a heating element.

In yet another example the expansion of the atrium support device 100 is performed by use of supplying a gas or liquid to the atrium support device 100. The use of the liquid for expanding the atrium support device 100 allows for water or blood to inflate the atrium support device 100 comprising an inflatable member reducing any complications if a leak of the atrium support device 100 would occur. If using gas to inflate the atrium support device 100 comprising the inflatable member the atrium support device 100 is inflated by using accessible means during heart surgery.

The present invention has been described above with reference to specific examples. However, other examples than the above described are equally possible within the scope of the invention. Different method steps than those described above may for instance be provided within the scope of the invention. The different features and steps of the invention may be combined in other combinations than those described. The scope of the invention is only limited by the appended patent claims.

1. A temporary atrium supporting device for delivery with a catheter, comprising:
an expandable and contractible intra atrial support member being resiliently flexible to allow for atrium contraction and expansion, when positioned intra atrial substantially maintaining atrial displacement volume of the beating heart, further comprising means for guiding an annuloplasty device from an insertion site to a securing site at a heart valve, and wherein said means for guiding is mechanically connected to the temporary atrium support.

2. The atrium support device according to claim 1, wherein the atrium support device comprises a plurality of atrium support members.

3. The atrium support device according to claim 1, wherein the atrium support member is an expandable cage, a wire, an inflatable member comprising at least one channel and/or an expandable covering with openings which allows for substantially maintaining the atrial displacement volume.

4. The atrium support device according to claim 1, wherein the atrium support member comprises an inlet and outlet for blood flow.

5. The atrium support device according to claim 1, wherein the atrium support member comprises a memory shape material, wherein the memory shape material has a first shape when deployed and a second expanded shape activated by a shape memory temperature.

6. The atrium support device according to claim 1, wherein the atrium support member comprises a heat set shape, and
wherein the atrium support member elastically returns to the heat set shape.

7. The atrium support device according to claim 1, further comprises means for aligning the atrium support device by use of at least one commissure, wherein the means for aligning comprises a first end and a second end, and
wherein the first end of the means for aligning is connected to the atrium support member and the second end is directed outwards from the atrium support member towards the at least one commisure.

8. The atrium support device according to claim 7, wherein the means for aligning are a pair of projections, projecting outwards from the atrium support member.

9. The atrium support device according to claim 1, wherein the atrium support member is partly in contact with the atrium.

10. The atrium support device according to claim 1, wherein the atrium support member is in contact with substantially the entire atrium.

11. The atrium support device according to claim 1, wherein the atrium support member is bent, banana shaped, spherical and/or bulb shaped when expanded.

12. The atrium support device according to claim 1, wherein the means for guiding is at least one ring shaped member arranged at an outer surface of the atrium support member.

13. The atrium support device according to claim 1, wherein the means for guiding is a channel arranged along an outer surface of the atrium support member.

14. The atrium support device according to claim 1, wherein the means for guiding is a plurality of holes at the atrium support member.

15. The atrium support device according to claim 1, wherein the means for guiding is at least one channel arranged through the atrium support member.

16. The atrium support device according to claim 1, wherein said atrium support member is resiliently flexible in an expanded state of the atrium support member.

17. The atrium support device according to claim 1, wherein the atrium support member has a predefined maximum expanded cross-section.

18. The atrium support device according to claim 1, wherein the atrium support member has a predefined minimum contracted cross-section when placed in the atrium, such that the compressive force exerted by the atrium on the atrium support member at the minimum contracted cross-section is compensated and counter acted by a reaction force of the support member on the atrium, the reaction force being equal to that of said compressive force, wherein said reaction force at the minimum contracted cross-section is set to a pre-defined value.

19-25. (canceled)
